(19) United States
(12) Patent Application Publication
(10) Pub. No.: US 2005/0143664 A1
(43) Pub. Date: Jun. 30, 2005

(54) Scanning probe using MEMS micromotor for endosocopic imaging

(76) Inventors: Zhongping Chen, Irvine, CA (US); Peter Tran, Irvine, CA (US)

Correspondence Address: Daniel L. Dawes;MYERS DAWES ANDRAS & SHERMAN LLP, 19900 MacArthur Boulevard, Suite 1150, Irvine, CA, 92612, US

(21) Appl. No.: 10/961,653

(22) Filed: Oct. 8, 2004

Related U.S. Application Data
(60) Provisional application No. 60/509,965, filed on Oct. 9, 2003.

(51) Int. Cl. A61B 6/00

(52) U.S. Cl. 600/478

(57) ABSTRACT

An endoscopic probe is combined with a source of radiation to measure a sample. A probe body includes a nonrotating transmission path and is communicated to the source to transmit radiation from the source from the proximal to the distal portion of the probe body. A micromotor is disposed in a distal portion of the probe body to provide a motive force. A movable scanner is coupled to the motor and is arranged and configured so that the scanner is directed toward or faces the transmission path. The scanner redirects the radiation from the source from the distal portion of the probe body into a scanned pattern onto the sample according to the motive force applied to the scanner from the motor. Back reflected radiation is received from the sample and is transmitted along the transmission path to the proximal portion of the probe body.

### RELATED APPLICATIONS

The present application is related to U.S. Provisional Patent Application Ser. No. 60/509,965, filed on Oct. 9, 2003, which is incorporated herein by reference and to which priority is claimed pursuant to 35 USC 119.

### GOVERNMENT SUPPORT

This invention was made with Government Support under Grant No. BES-0086924, awarded by the National Science Foundation. The Government has certain rights in this invention.

### BACKGROUND OF THE INVENTION

1. Field of the Invention

The invention relates to the field of endoscopic probes and in particular to endoscopic probes used for optical coherence tomography (OCT).

2. Description of the Prior Art

Direct visualization of tissue anatomy and physiology provides important information to the physician for the diagnosis and treatment of disease. Noninvasive techniques with high spatial resolution for tomographic imaging of in vivo tissue structure and physiology are currently not available as a diagnostic tool in clinical medicine. Such techniques could have a significant impact for biomedical research and patient treatment. Techniques such as ultrasound and Doppler ultrasound are currently used to image tissue structure and blood flow. However, the relatively long acoustic wavelengths limit the spatial resolution to approximately 100 μm.

Optical coherence tomography (OCT) is a recently developed imaging modality based on coherence-domain optical technology. OCT takes advantage of the short coherence length of broadband light sources to perform micrometer-scale, cross-sectional imaging of biological tissue. OCT is analogous to ultrasound B-mode imaging except that it uses light rather than sound. The high spatial resolution of the OCT structural image enables noninvasive in vivo “optical biopsy” and provides immediate and localized diagnosis information. A number of extensions of OCT capabilities for functional imaging of tissue physiology have been developed.

Optical Doppler tomography (ODT), for example, combines the Doppler principle with OCT to obtain high resolution tomographic images of tissue structure and blood flow simultaneously. Spectroscopic OCT combines spectroscopic analysis with OCT to obtain the depth resolved tissue absorption spectra.

Polarization sensitive OCT (PS-OCT) combines polarization sensitive detection with OCT to determine tissue birefringence. F-OCT provides clinically important information on tissue physiology such as tissue blood perfusion, oxygen saturation, and hemodynamics in addition to tissue structure. It has a number of potential clinical applications such, as vasoactive drug screening, tissue viability and burn depth determination, tumor angiogenesis studies, and bleeding ulcer management.

OCT was first used clinically in ophthalmology for the imaging and diagnosis. of retinal disease. Recently, it has been applied to imaging structure in skin, vessels, oral cavity as well as respiratory, urogenital, and GI tracts. The first in vivo endoscopic OCT images in animals and humans were reported in 1997. Since then, a number of clinical applications for endoscopic OCT imaging of respiratory, urogenital, and GI tracts have been reported by a number of groups [22-32]. The potential applications of endoscopic OCT to GI tract is particularly interesting because many common GI lesions occur within the imaging depth of the OCT system (1-2 mm).

Most of the current endoscopic OCT's use a mechanical transducer such as a cable to move a prism mounted in a distal probing tip to scan the beam. The design is similar to commercial endoscopic ultrasound where scans are provided by a remote motion actuator that uses a cable as a mechanical transducer to convey the motion through the probe. In the proximal end of the endoscope, a motor was used to drive the distal end to create the scanning patterns. The coupling of the torque is performed by means of a steel cable. The distal end is composed of a gradient index lens (GRIN) coupled together with a prism and a single mode fiber. The prism is linked directly or indirectly to the GRIN lens to divert the light toward the tissue. In the traditional design, the optical fiber, GRIN lens and prism rotate usually rotate together. At the present time users have started to make angle polish GRIN lenses at the interface of the optical fiber and GRIN lens to reduce back reflection. This prior art design requires a rotary fiber optical coupling joint.

A gradient index lens (GRIN) is used to focus and collimate light sources. It is widely used in both active and passive fiber-optics components, MEMS and imaging systems. When light rays travels between air and glass, it will change its direction according to the change of index of refraction of the traveled medium. A conventional lens focuses a light beam by bending lights at its surface through controlling the lens shape and smoothness of its surface. Unlike conventional lenses, GRIN lenses focus light by gradually varying the index of refraction within the lens material, rather than the thickness, of the optical element. Through a precisely controlled radial variation of the index of refraction of the material of the lens from the optical axis to the edge of the lens, a GRIN lens can smoothly and continually redirect light beam to point of focus without the need to tightly-control the surface curvature.

There are a number of limitations in the current endoscopic OCT probe design. First, the rotary fiber optical coupling joints are difficult to make, and significant loss can occur in the joint. Second, nonuniform coupling efficiency is generated when the fiber is rotated. Third, the friction between the outer stationary sheath and inner rotating sleeve usually create a nonuniform rotational torque as the endoscope is bent as it transverses through the body which produces a nonuniform scanning speed.

What is needed is a design for endoscopic OCT probe which overcomes each of the limitations of the prior art without introducing additional limitations or costs.

### BRIEF SUMMARY OF THE INVENTION

The invention is an endoscopic probe for use in combination with a source of radiation for measurement of a sample comprising a probe body having a proximal and distal portion, a nonrotating transmission path disposed in the probe body communicated to the source for conducting radiation from the source from the proximal to the distal portion of the probe body; a motor disposed in a distal portion of the probe body to provide a motive force; and a movable scanner coupled to the motor and arranged and configured so that the scanner is directed toward or is facing the transmission path. The scanner redirects the radiation from the source from the distal portion of the probe body into a scanned pattern onto the sample according to the motive force applied to the scanner from the motor. Back reflected radiation is received back from the sample and is transmitted along the transmission path to the proximal portion of the probe body, where it can then be utilized in a detector or sensing system.

In the illustrated embodiment the source of radiation comprises a laser, the transmission path comprises a stationary optic fiber coupled to a GRIN lens, the motor comprises a microelectromechanical systems (MEMS) motor, and the scanner comprises a prism or mirror coupled to the motor. The mirror/prism and MEMS motor may be fabricated as an integrated unit. The motor rotates or oscillates the prism.

The motor is arranged and configured in an inverted configuration to allow the direct reflection of radiation from the scanner onto the sample. In this manner, the scanner is directly optically communicated to the GRIN lens. The optic fiber is provided with a tapered tip or combined with a pinhole or other optical means to increase the optical resolution.

The fiber optic may be replaced by a plurality of optical fibers to allow different input fiber type signals, including a single mode fiber, a fiber bundle, a multimode fiber, a group of tapered single mode fibers.

The endoscopic probe may further comprise the source and a separate detector of the radiation.

The endoscopic probe further also comprise a source of RF energy and an antenna coupled to the motor, so that the motor is remotely powered by the source of RF energy.

In the illustrated embodiment the endoscopic probe further comprises an optical coherence tomographic (OCT) system having a sample probe wherein the endoscopic probe is coupled and employed as the sample probe.

The radiation used in the endoscopic probe is not restricted to light or electromagnetic radiation, but the source of radiation may comprise a source of ultrasound, the transmission path may comprise a stationary acoustic channel, the motor may comprise a MEMS motor, and the scanner may comprise an ultrasound deflector coupled to the motor.

The endoscopic probe may further comprise an actuator coupled to the motor to selectively move the focal point of the radiation redirected from the scanner into the sample by linearly displacing the motor as its rotates the scanning prism or mirror to obtain a three-dimensional OCT image. The actuator is coupled to the scanner to change the distance between the transmission path and scanner to selectively move the focal point of the radiation which is redirected from the scanner into the sample for high resolution OCT with focus tracking. In the illustrated embodiment the actuator is connected to the motor and linearly displaces the motor to controllably change the distance between the GRIN lens and the prism or mirror.

The endoscopic probe is characterized by the fact that the proximal portion of the endoscopic probe is provided only with the transmission path and does not require a rotational coupler of any kind.

The endoscopic probe may further comprise a gearhead coupled to the motor to reduce angular rate output of the motor.

The invention is also expressly defined as a method by which the above disclosed endoscopic probe operates.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a cut-away side plan view of an endoscope according to the invention.

FIG. 2 is a diagrammatic block diagram of an OCT scanning system in which the endoscope of FIG. 1 is employed.

FIG. 3A is a photograph of the assembled endoscope of the invention.

FIG. 3B is an enlarged photograph of the rotating prism and MEMS motor used in the endoscope of the invention.

FIG. 3C is a series of photographic frames illustrating the scanning of light from the assembled endoscope of the invention as indicated by the arrowhead.

FIG. 4 is a graph of the signal-to-noise ratio in the optical signal at each of the optical interfaces in the optical path of the endoscope of the invention.

FIG. 5A is a photograph of a scanned image of an in vitro rabbit trachea using a prototype of the endoscope of the invention.

FIG. 5B is a photograph of a scanned image of an in vivo rabbit esophagus using a prototype of the endoscope of the invention.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

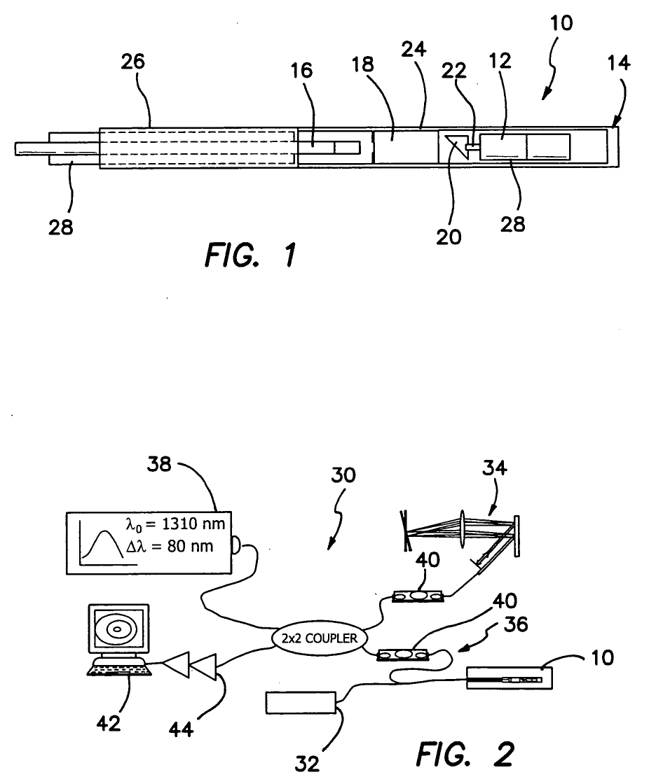

The current invention overcomes each of the limitations of the prior art by using a microelectromachined silicon (MEMS) motor 12 near the tip 14 of the endoscope 10 to perform the scanning. No driving cable is required or used. A side cut-away plan view of the distal end portion of the illustrated embodiment of the endoscope 10 is shown in FIG. 1. The design of endoscope 10 is much simpler than the prior art designs. A single mode optical fiber 16 with or without a tapered tip is mounted to the GRIN lens 18 at the appropriate distance. A prism 20, which has a diameter or radial envelope within endoscope 10 smaller than the diameter of motor 12 is mounted on a shaft 22 or otherwise coupled to motor 12. In the preferred embodiment motor 12 provides a full rotary motive force or torque applied through shaft 22 to the prism 20. However, it is expressly contemplated that motor 12 will also supply an oscillatory motive force or torque to prism 20 through a predetermined angular interval.

Prism 20 is disposed in an inverted position to reflect the light from GRIN lens 18 through a transparent window 24 defined in sheath 26 into the tissue or environment surrounding tip 14 of endoscope 10. The outer sheath 26 makes mechanical contact with and provides mechanical support for the micromotor outside covering 28, GRIN lens 18, and optical fiber 16. Optical fiber 16 is preferably centered in endoscope 10 and supported by an axially concentric sleeve 28 of flexible plastic, metal or other spacing material. The invention also contemplates the embodiment where no sleeve 28 is present and the optical fiber is free-floating in an air filled lumen.

An electrical connection is routed down the side of the motor 12 and is powered by radio frequency from an external power source (not shown) which is located proximally from tip 14. In addition, wireless power delivery to motor 12 is possible since the power required for the MEMS motor 12 is extremely small. The entire diametric size of endoscope 10 is similar to or smaller than the present ultrasound endoscope systems in use.

Although disclosed design is made for optical coherence tomography, OCT, the design of the invention can also be applied to other imaging devices such as fluorescence, thermal, and other radiation imaging with the appropriate modifications according to well known design principles that require an endoscopic scanning probe.

For example, consider the following alternative embodiments and usages of the endoscope design. First, the prism 20 may be replaced by an ultrasound deflector and the inputting fiber 16 with a matching ultrasound source and sensor. The disclosed inverting motor configuration is then used to scan the image for ultrasound. Second, the scanning probe 10 may operatively scan with a different optical technology or scheme, such as second harmonic or two photon detection and scanning optics. Third, dynamic focus tracking can be achieved by adding a crystal or small electronic actuator (not shown) to shift the shaft 22 to move the focal point, which will increase the spatial resolution over a larger depth range. Fourth, the motor 12 and any other electrical circuitry in the probe 10 may be remotely powered by an RF field pickup coil or antenna, so that there is no direct wiring to the motor 12 in the endoscopic probe 10.

Thus, it can now be appreciated that the invention is characterized by a motor 12 that provides the torque is at the distal end of the endoscope 10. This creates a more constant rotation and reduces image distortion due to a changing frame rate. The inputting fiber 16 does not rotate at its proximal or distal base to scan as in the prior art ultrasound or OCT probe. The motor 12 with and without gears uses an inverted configuration, i.e. has the motive end directed proximally instead of distally, to allow the direct reflection of light from the GRIN lens 18 toward the tissue. If the motor 12 is not mounted backwards, a surface would be needed to back reflect the light, which surface would be much harder to make. The coupling in endoscope 10 used for scanning is performed immediately after the GRIN lens 18 which improves signal quality. The rotating prism 20 is positioned in the strongest part of the optical light beam to reduce noise. Placing the motor 12 immediately adjacent to GRIN lens 18 optimizes performance, because lens 18 serves to enlarge the core of the light beam. A smaller six micrometer tapered tip diameter of optical fiber 16 can be used to increase the optical resolution. A pinhole can also be used in the optical path at the distal end of an endoscope 10 for the purpose of increasing resolution. Currently, the single mode fiber 16 is about 9 micrometers in diameter. If we decrease this diameter by half, we increase the resolution by a factor of two. Thus, we want the smallest diameter possible for the scanning aperture, so that invention includes the coupling of an optical device with the single mode fiber 16 that serves to reduce its diameter, such as a sharpened tip, pin hole or the like, to increase the resolution. The input optical path is fixed to allow different input fiber types of signals, such as one single mode fiber, a fiber bundle, a multimode fiber, a group of tapered single mode fibers, or even a separate light source and detector to be selected as the optical input source. Endoscope probe 10 can be used in the various types or modes of optical scanning, such as OCT, two photons, second harmonics, since the scanning principle is similar in each.

An illustrated example of the disclosed endoscopic optical coherence tomography probe 10 was fabricated using a 1.9 mm microelectromechanical system (MEMS) motor 12. The design of the MEMS endoscope 10 eliminates the need to couple the rotational energy from the proximal to distal end 14 of the endoscope 10. Furthermore, the endoscope's body or outer sheath 26 has the advantages of being much smaller and more flexible than the traditional endoscopes since no reinforcement is needed to couple the rotational torque.

At the distal portion of endoscope 10, prism 20 was mounted on micromotor 12 to deflect the light rays to create a transverse circular scanning pathway. Because of the MEMS scanning, the optical signal is more stable with the single mode fiber 16 being stationary.

Endoscope 10 is connected to a conventional OCT system as diagrammatically depicted in the block diagram of FIG. 2. As shown in FIG. 2, the basic principle behind OCT is a Michelson interferometer 30. FIG. 2 is a schematic of OCT system with MEMS probe 10 and a controller motor controller 32. The OCT signal amplitude is determined by the interference fringe created between a conventional fast scanning Fourier-domain optical delay reference line 34 and the sample arm 36. The laser 38 used for the system 30 was a traditional superluminescent diode (SLD) centered at 1310 nm with a width of 80 nm. In addition, phase modulators 40 were placed in the reference arm 34 and sample arm 36 to compensate for the various polarizations and to reduce back reflection. The data was digitalized at 5 MHz by analog-to-digital converter 44 and signal processing was done in computer 42 as described previously using a digital approach to generate an analytical OCT signal. Other OCT configurations could be equivalenty substituted, such as frequency domain OCT, spectral domain OCT, Doppler OCT, spectroscopic OCT, polarization sensitive OCT, functional OCT and related OCT protocols as may be now known or later devised.

For the endoscopic portion of system 30 a synchronous controller 32 powers the MEMS motor 12 through a three phase AC signal. The schematic for the MEMS endoscope is shown in FIG. 1 as discussed above. Unlike a traditional catheter-endoscope, the MEMS endoscope 10 has a much simpler proximal and body design. It completely eliminates the need to precisely align the fixed fiber 16 with the rotational drive shaft 22. There is no male-to-male, or male-to-female, axially flexible shafts, or gradient-index (GRIN) lens at the proximal end of endoscope 10. All scanning operations are performed at the distal portion of endoscope 10 with MEMS motor 12 and micro-optical components 16, 22, 24. Because the fiber 16 is not rotating in the body of the endoscope 10, no metallic sleeve for reinforcement is necessary. For the MEMS design, the body is composed of a single mode fiber 16 with three-twisted wires (not shown) enclosed in a biocompatible polytetrafluroethylene (PTFE) tube. In the illustrated embodiment, a 4 mm GRIN lens 18 with a diameter of 1 mm was used since it was readily available with a 60 and 30 angle cleavage. The radial scanning is done at the distal portion of endoscope 10 with a 1.9 mm MEMS motor 12 coupled to a 0.7 mm prism 20. The MEMS micromotor 12 is mounted is a backward configuration facing the proximal end of endoscope 10 and prism 20 is used to deflect the optical light toward the sample.

In the illustrated embodiment a 49:1 gearhead was added to slow the motor 12 down and create a more uniformed rotational speed. By itself, the Faulhaber MEMS motor 12 can achieve a speed of 1.5 kHz; but with the gearhead in place, the maximum achievable speed is around 30 Hz. The outside circumference of the probe 10 was 6.3 mm (diameter of 2 mm) and it was not possible to scan at the 30 Hz rate so the scanning rate was set to under 1 Hz.

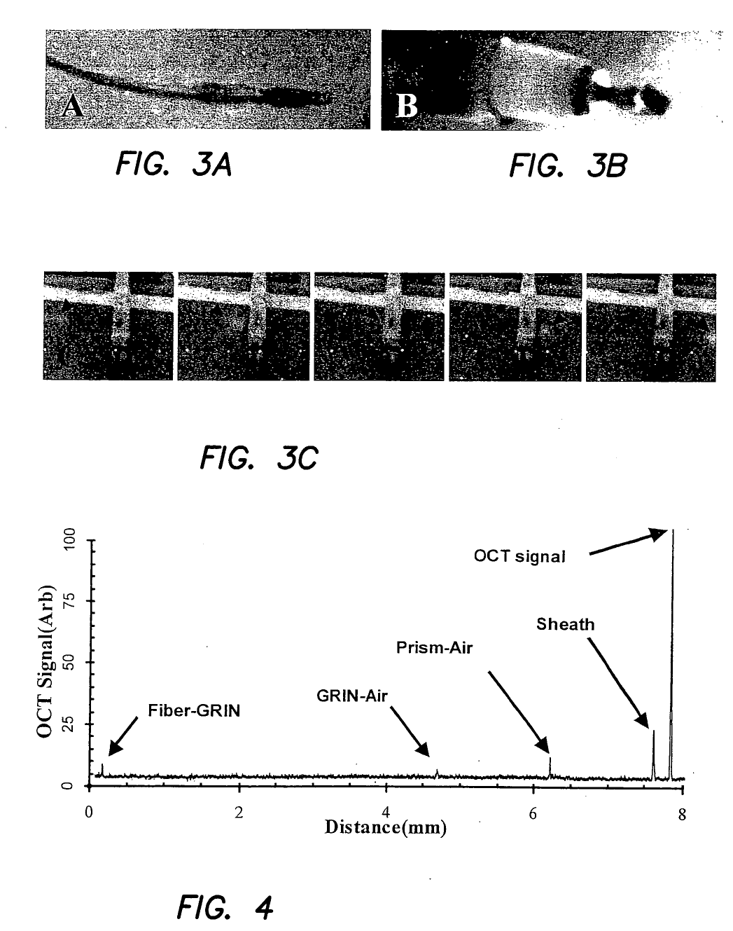

A photograph of endoscope 10 is shown in FIG. 3A with a close-up photograph of motor 12 and prism 20 with the outer sheath 26 removed shown in FIG. 3B. FIG. 3C shows a series of photographic frames of the endoscope 10 on top of an infrared card in a movie style format. As the motor shaft 22 rotates, the luminescent on the infrared card moves from left to right as indicated by the arrowheads in each frame of FIG. 3C. The arrowheads show the movement of the beam as shaft 22 rotates and illustrates the power of the back reflected light since this is the source of most of the problems found in conventional OCT endoscopes. After optimization, the highest back reflected noise is from the PTFE sheath 26 and not the optic components.

The signal at the GRIN lens-to-fiber interface is reduced by using an angle polished fiber along with an angled GRIN lens 18. Further reduction at the GRIN lens-to-fiber interface was accomplished using an ultra violet adhesive to eliminate the air and glass mismatch index of reflection. The distance between the GRIN lens and fiber face were dynamically determined by measuring the returning optical power. The working distance was set at the middle of the image or 2 mm outside the PTFE tubing 26 (4 mm from the end of the GRIN lens 18). Further down the endoscope 10 is the GRIN lens-to-air interface. The internal backreflection at this interface is usually lower than the fiber-to-GRIN lens interface because the light coming out of the GRIN lens 18 is angled relative to the surface normal. Nevertheless, a 3-degree angle is used to ensure low back reflection. The back reflection at the prism-to-air interface is somewhat larger because the light approaches the normal as it goes toward the focal point, but the back reflected signal is still less than 5%.

After the prism 20 in the optical path is the enclosure of the PTFE sheath 26 and the OCT signal itself. In the illustrated embodiment, a medical grade PTFE tube 26 was used to enclose the prism 20 and GRIN lens 18. If everything is done correctly, a strong OCT signal is obtained which saturated the detector as shown in the graph of FIG. 4 when an IR card or mirror is used as a reference. FIG. 4 is a graph showing the signal-to-noise ratio of the endoscope 10 at different optical interfaces in the optical path down endoscope 10. As shown by the FIG. 4, over 90% of the back reflected photonic energy is from the sample itself and is in the OCT signal.

In vivo esophageal data and in vitro trachea data were taken with the MEMS probe 10. For in vivo imaging, New Zealand white rabbits (2.3-4.8 kg) were anesthetized with a 2:1 mixture of ketamine HCl (100 mg.mL): Xylazine (20 mg/L) at a dose of 0.75 mL/kg through a 20 gauge catheter in the marginal ear vein. Respiration rate were maintained at a rate of 30 to 40 breaths per minute and at a tidal volume of 50 mL through a 3-mm endotracheal tube using a Harvard Apparatus Dual Phase Control Respiratory Pump. A mixture of 1:1 ketamine HCl (100 mg/mL): xylazine (20 mg/mL) ear were given as necessary to maintain anesthesia. For in vitro testing, a piece of trachea was taken from a euthanized rabbit. The trachea were cut vertically and wrapped around the endoscope 10. An image was taken using a standard linear scanning and converted to cylindrical format using Matlab software.

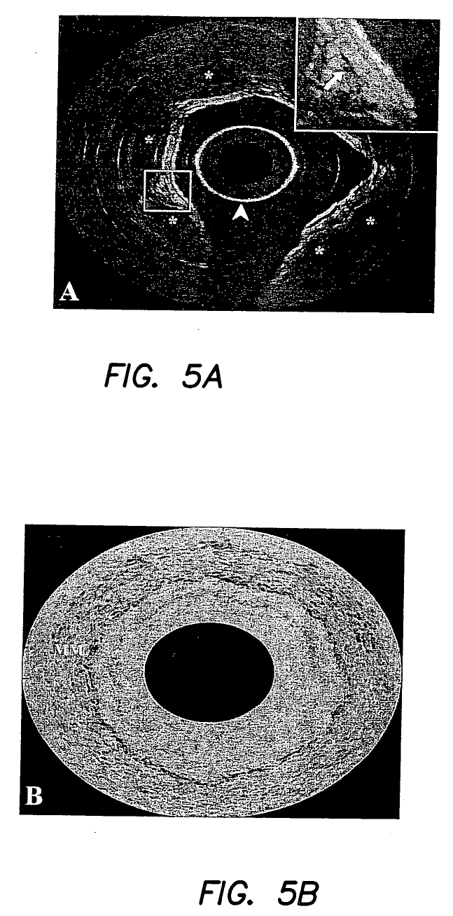

The in vitro data of the rabbit trachea is shown in the photograph of FIG. 5A. FIG. 5A is an in vitro image of the rabbit trachea wrapped around the endoscope 10. Trachea cartilages denoted by an asterisk * and glands denoted by the arrow in the top right inset can be seen in FIG. 5a. The arrowhead indicates the 2 mm PTFE tubing. FIG. 5B is an in vivo image of the esophagus. Inner black circle has a diameter of 3 mm. The trachea cartilage ring can be seen beneath the epithelial. Different layers as mucosa and submucosa can also be seen. The resolution for FIG. 5B is much lower since the tissue farther from the PTFE tubing and the protocol is not optimized for in vivo recording. Nevertheless, the muscularis mucosae (MM) can be seen as a dark band.

The resolution for the MEMS endoscope system is about 13 micrometer at the PTFE enclosure 26 and decreases away from endoscope 10. Unlike a linear scanning endoscope, which produces constant sample rates throughout the tissue depth, a rotational endoscope's resolution decreases away from the origin. For FIG. 5A, the resolution varies from 13 micrometer at the PTFE sheath 26 to 40 micrometers at the border of the image. The potential to obtain much better resolution for rotational MEMS endoscope OCT probes 10 is present because a MEMS probe offers the possibility of circular scanning using the high rotational speed of the Faulhaber motor. The data acquisition process is not optimized in the illustrated embodiment, although in principle the design of the invention would allow it, and the image quality in illustrated embodiment deteriorates considerably when used for an in vivo experiment. This limitation is an artifact of the prototype and is not an inherent limitation in the invention which can be significantly improved over that demonstrated in the illustrated embodiment which is only an initial experimental prototype. The tissue in FIG. 5B has a circumference of 20 mm (6 mm diameter), and the resolution drops by a factor of 4 just to keep a sampling rate of 1 Hz. Furthermore, the tissue is beyond the 2 mm focal point.

What is illustrated in FIGS. 2-5B is a MEMS probe 10 which can be used to image in vitro rabbit trachea and in vivo esophageal. As MEMS technology develops further, this type of endoscope will become a preferred method and apparatus to image tissue since it provides a stable radial signal compared to conventional rotational techniques.

Many alterations and modifications may be made by those having ordinary skill in the art without departing from the spirit and scope of the invention. For example,

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

1. An endoscopic probe for use in combination with a source of radiation for measurement of a sample comprising:
a probe body having a proximal and distal portion;
a nonrotating transmission path disposed in the probe body communicated to the source for conducting radiation from the source from the proximal to the distal portion of the probe body;
a motor disposed in a distal portion of the probe body to provide a motive force; and
a movable scanner coupled to the motor and arranged and configured so that the scanner is directed toward the transmission path, the scanner redirecting the radiation from the source from the distal portion of the probe body in a scanned pattern onto the sample according to the motive force applied to the scanner from the motor and receiving back reflected radiation from the sample to be transmitted along the transmission path to the proximal portion of the probe body.

2. The endoscopic probe of claim 1 where the source of radiation comprises a laser, where the transmission path comprises a stationary optic fiber coupled to a GRIN lens, where the motor comprises a MEMS motor, and where the scanner comprises a prism or mirror coupled to the motor.

3. The endoscopic probe of claim 2 where the motor rotates the prism or mirror.

4. The endoscopic probe of claim 2 where the motor oscillates the prism or mirror.

5. The endoscopic probe of claim 1 where the motor is arranged and configured in an inverted configuration to allow the direct reflection of radiation from the scanner onto the sample.

6. The endoscopic probe of claim 2 where the scanner is directly optically communicated to the GRIN lens.

7. The endoscopic probe of claim 2 where the optic fiber is provided with a tapered tips to increase the optical resolution.

8. The endoscopic probe of claim 2 further comprising a pinhole and where the distal end of the optic fiber optically communicates with the pinhole to increase the optical resolution.

9. The endoscopic probe of claim 1 further comprising optical means communicated to the transmission path to reduce optical beam diameter and to increase resolution of the endoscopic probe.

10. The endoscopic probe of claim 2 where the fiber optic comprises a plurality of optical fibers to allow different input fiber type signals, including one single mode fiber, a fiber bundle, a multimode fiber, a group of tapered single mode fibers.

11. The endoscopic probe of claim 1 further comprising the source and a separate detector of the radiation.

12. The endoscopic probe of claim 1 further comprising a source of RF energy and an antenna coupled to the motor, so that the motor is remotely powered by the source of RF energy.

13. The endoscopic probe of claim 1 further comprising an optical coherence tomographic (OCT) system having a sample probe wherein the endoscopic probe is coupled and employed as the sample probe.

14. The endoscopic probe of claim 1 where the source of radiation comprises a source of ultrasound, where the transmission path comprises a stationary acoustic channel, where the motor comprises a MEMS motor, and where the scanner comprises an ultrasound deflector coupled to the motor.

15. The endoscopic probe of claim 1 further comprising an actuator coupled to the scanner to change the distance between the transmission path and scanner to selectively move the focal point of the radiation which is redirected from the scanner into the sample for high resolution OCT with focus tracking.

16. The endoscopic probe of claim 1 where the proximal portion of the endoscopic probe has no rotational coupling in the transmission path.

17. The endoscopic probe of claim 1 further comprising a gearhead coupled to the motor to reduce angular rate output of the motor.

18. A method of operating an endoscopic probe in combination with a source of radiation for measurement of a sample comprising:
transmitting radiation along a nonrotating transmission path disposed in a probe body communicated to the source from the source from the proximal to the distal portion of the probe body;
providing a motive force from a motor disposed in a distal portion of the probe body to a scanner;
redirecting the radiation from the source by a proximally directed, movable scanner from the distal portion of the probe body in a scanned pattern onto the sample according to the motive force applied to the scanner from the motor; and
receiving back reflected radiation from the sample to be transmitted along the transmission path to the proximal portion of the probe body.

19. The method of claim 18 where the motor rotates the scanner.

20. The method of claim 18 where the motor oscillates the scanner.

21. The method of claim 18 where the motor is arranged and configured in an inverted configuration to allow the direct reflection of radiation from the scanner onto the sample.

22. The method of claim 18 where the scanner comprises a MEMS mirror and where providing a motive force comprises rotating the MEMS mirror with a MEMS motor integrated with the MEMS mirror.

23. The method of claim 18 where the scanner comprises a MEMS scanner integrated with the motor and where providing a motive force comprises linearly displacing the rotating scanner to obtain a three dimensional OCT image.

24. The method of claim 18 further comprising remotely powering the motor from a wireless energy source.

25. The method of claim 18 further comprising performing optical coherence tomography (OCT) on back reflected radiation from the sample.

26. The method of claim 18 further comprising performing ultrasound tomography on back reflected radiation from the sample.

27. The method of claim 18 further comprising selectively moving the focal point of the radiation redirected from the scanner into the sample.
